(19) United States
(12) Patent Application Publication
(10) Pub. No.: US 2014/0371673 A1
(43) Pub. Date: Dec. 18, 2014

(54) BALLOON CATHETER

(71) Applicant: QualiMed Innovative Medizinprodukte GmbH, Winsen/Luhe (DE)

(72) Inventor: Martina Maria Schmitt, Garstedt (DE)

(73) Assignee: QUALIMED INNOVATIVE MEDIZINPRODUKTE GMBH, Winsen/Luhe (DE)

(21) Appl. No.: 14/374,367

(22) PCT Filed: Jan. 23, 2013
(86) PCT No.: PCT/EP2013/051182

§ 371 Date: Sep. 5, 2014

(30) Foreign Application Priority Data
Jan. 24, 2012 (DE) 10 2012 001 188.1

(51) Int. Cl. A61M 25/10; A61L 29/16; A61L 29/08

(52) U.S. Cl. CPC A61M 25/1002; A61M 25/104; A61L 29/08; A61L 29/16; A61M 2025/1086; A61L 2300/416; A61L 2300/606
USPC 604/103.02; 606/194

(57) ABSTRACT

The invention relates to a balloon catheter with a catheter body (1) and with a balloon (2), wherein the balloon (2) is arranged along part of the length of the balloon catheter and can be expanded by delivery of a pressure medium through a delivery line (3). The balloon catheter is characterized in that the outside of the balloon (2) has a multiplicity of punctiform or groove-shaped depressions (4). These improve the application of forces to a vascular wall or stent surrounding the balloon (2). Moreover, in the case of balloons (2) coated with a medicament, the adherence of the medicament is strengthened.

The invention relates to a balloon catheter having a catheter body and a balloon, wherein the balloon is disposed along part of the length of the balloon catheter and can be inflated by means of supplying a pressure medium by way of a feed line.

Balloon catheters are known and serve, within the scope of angioplasty, for expanding a blood vessel, for example one that has been constricted due to arteriosclerotic deposits. For this purpose, the balloon catheter is introduced into the vascular system, and the balloon, which is situated in the distal region of the balloon catheter, is brought up to the constriction of the blood vessel in the relaxed state. Subsequently, the balloon is inflated by means of a pressure medium, in order to thereby eliminate the vascular constriction. Furthermore, stents are frequently implanted, which are supposed to bring about the result that the blood vessel remains permanently open (stent angioplasty).

In order to prevent renewed constriction (re-stenosis) of the blood vessel, in the meantime balloons are frequently used that are additionally coated with a medication, which is delivered to the blood vessel wall when the balloon is inflated. Currently, the cytostatic paclitaxel is particularly used; another medication that can be used is rapamycin.

In the medication-coated balloon catheters that have been used until now, it has frequently been noted as a disadvantage that the amount of medication that is delivered at the treatment site is not sufficient. For this reason, the task arises of improving the adhesion of the medication to the balloon and of increasing the amount of medication that can be applied, so that a sufficient supply to the blood vessel wall to be treated is ensured.

This task is accomplished, according to the invention, by means of a balloon catheter having a catheter body and a balloon, wherein the balloon is disposed along part of the length of the balloon catheter and can be inflated by means of supplying a pressure medium by way of a feed line, and wherein the balloon has a plurality of point-shaped or groove-shaped depressions on its outside.

It has surprisingly been shown that the adhesion of the medication on the outside of the balloon is clearly improved if this outside has depressions. Obviously, an increased amount of medication can collect in the depressions. Furthermore, it has been shown that amounts of medication situated in the depressions remain on the balloon better, for example during the course of storage or during introduction of the balloon into the vascular system. The delivery of medication in regions of the blood vessel in which no treatment is required is also minimized, accordingly.

In addition, it has been shown that a balloon surface having depressions also improves the transfer of forces to the blood vessel wall. Presumably, this is attributable to the fact that the forces are exerted, in the case of a structured surface, more by individual, projecting regions of the balloon surface. By means of the reduction of the direct contact surface with the blood vessel wall, the pressure in these regions (pressure=force per surface area) is increased. In this manner, a stronger effect is exerted on the blood vessel wall than in the case of a smooth surface. The effect is also reinforced during expansion of a stent, since the structured surface of the balloon “hooks into” the braid structure of the stent. Furthermore, the connection between stent and balloon counteracts undesirable contraction of the stent, in that the individual parts of the stent are held in position more strongly. During expansion, the stent therefore tends less toward contraction and instead, more to stretching of the expansion elements situated in the braid structure. These effects are achieved even if the balloon is not coated with a medication.

Any therapeutically practical medication can be used as a medication. A mixture of multiple active substances can also be involved. In particular, those medications that inhibit cell proliferation are involved, in order to prevent the dilated region of the blood vessel from being stenosed, once again, by cells growing into it. Coating of balloons is fundamentally known in the state of the art; the corresponding coating methods can be used. Examples of medications that can be used are: paclitaxel, rapamycin, everolimus, tacrolimus, zotarolimus, as well as corresponding structural derivatives.

Furthermore, the medication coating can contain ancillary substances. Examples of these are polyvinyl pyrrolidone (PVP), polysorbates or polyethylene glycol. A biocompatible plasticizer such as glycerol, polyethylene glycol or propylene glycol can also be contained in the medication coating.

It must be possible to fill the balloon with a pressure medium by way of a feed line, in order to bring about inflation in this way. This feed line typically runs through the interior of the catheter body, whereby the catheter body can also have multiple channels or concentrically disposed passages, if necessary. For example, a further channel that runs in the longitudinal direction can serve for passing a guide wire through. Inflation of the balloon takes place under high pressure, which typically lies between 6 and 20 bar.

The catheter body has an elongated shape, so that the balloon catheter can be introduced by way of the femoral artery, for example, and advanced to the stenosed site with X-ray monitoring. The end facing the physician, i.e. the end from which the physician advances the balloon catheter, is referred to as the proximal end; the opposite end is referred to as the distal end. Distal therefore corresponds to the advancing direction of the balloon catheter. The balloon is disposed in the region of the distal end of the balloon catheter. The balloon itself also typically has an elongated shape, so that dilation of the blood vessel and application of a medication can take place over a sufficient region.

One balloon variant has an inner passage for the surrounding bodily fluids, particularly blood. This can be, for example, what is called a tube-type balloon, where the tube is wound up in helical shape to form a dilatable wall, and is surrounded by the medication-permeable outer membrane. A wound-up tube-type balloon is described, for example, in WO 2007/012443 A1. The use of such a tube-type balloon having a central passage makes it possible to leave the balloon in a blood vessel for a certain period of time and thereby to extend the application time period for the medication.

The depressions applied to the outside of the balloon can particularly be structured in groove shape. In this connection, different configurations are possible; for example, the depressions can be configured circumferentially on the balloon surface, whereby the depressions can be oriented orthogonal to the longitudinal axis of the balloon.

A further possibility consists in that the groove-shaped depressions run in a helical line, in helix shape, on the outside of the balloon, wherein the longitudinal axis of the helix corresponds to the longitudinal axis of the balloon. In this case, the depressions and the projecting regions situated between them essentially form a screw thread. In this way, particularly tight profiling of the balloon surface is brought about. Also possible, however, is an embodiment in which the groove-shaped depressions run parallel to the longitudinal axis of the balloon or in sine shape on the outside of the balloon.

Aside from groove-shaped depressions, however, point-shaped depressions are also possible. Point-shaped depressions are understood to be those that have a round, oval, or also angular shape when viewed from above, for example.

It has proven to be particularly advantageous if the depressions have a depth of 1 to 20 μm, preferably of 3 to 8 μm, and particularly preferably of 4 to 6 μm. Such depressions are suitable, on the one hand, for accommodating a sufficient amount of medication and from storing it until the correct point in time, but on the other hand, the depth is also dimensioned in such a manner that when the balloon is inflated, the medication is effectively applied to the blood vessel wall. The aforementioned range has also proven to be advantageous with regard to exerting force on the blood vessel wall or on a stent.

Furthermore, it has been proven to be effective if the individual depressions have a distance between them of 200 to 400 μm, particularly of approx. 300 μm. This is particularly true in the case of groove-shaped depressions that are disposed essentially parallel. Both with regard to medication delivery and with regard to exertion of force, this has been shown to be the most suitable range. In this connection, the precise distance between the individual depressions can be the same or can lie within the bandwidth indicated. In this connection, one can more likely assume a regular distance in the case of groove-shaped depressions, while the distance between point-shaped depressions can vary.

The invention will be explained in greater detail using the attached figures. These show:

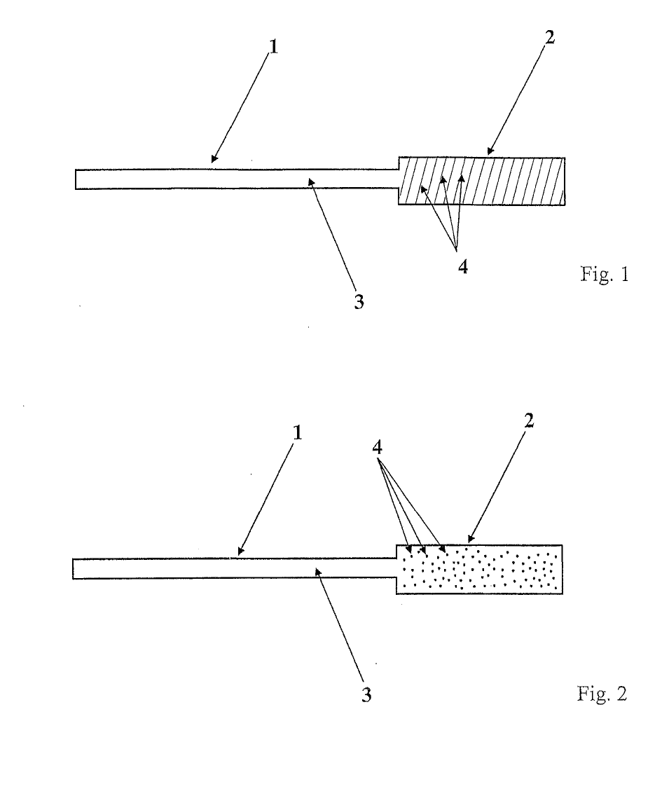

FIG. 1 a schematic representation of an exemplary embodiment of the balloon catheter according to the invention, and

FIG. 2 a schematic representation of a further exemplary embodiment of the balloon catheter according to the invention.

FIG. 1 schematically shows an exemplary embodiment of the balloon catheter 1 according to the invention in longitudinal section. In the representation selected here, left means proximal and right means distal. The balloon catheter 1 has a balloon 2, to which a pressure medium can be applied for inflating it within a stenosed region of a blood vessel. This pressure medium is introduced into the balloon 2 by way of the feed line 3. The surface of the balloon 2 is provided with a coating that contains a medication. This coating is applied to the blood vessel wall when the balloon 2 has been expanded.

On its surface, the balloon 2 has a plurality of groove-shaped depressions 4, which are disposed in the form of a screw thread in this exemplary embodiment. By means of the arrangement of the depressions 4, the result is achieved that a greater load of medication on the balloon surface is possible. Furthermore, the adhesion of the medication to the balloon surface and the exertion of force on the blood vessel or the stent surrounding the balloon 2 are also improved.

In FIG. 2, an alternative embodiment is shown, which differs from the embodiment according to FIG. 1 in that the depressions 4 are point-shaped and disposed on the surface of the balloon 2 in less regular manner.

1. Balloon catheter having a catheter body (1) and a balloon (2), wherein the balloon (2) is disposed along part of the length of the balloon catheter and can be inflated by means of supplying a pressure medium by way of a feed line (3),
wherein
the balloon (2) has a plurality of point-shaped or groove-shaped depressions (4) on its outside.

2. Balloon catheter according to claim 1, wherein the balloon (2) has a coating that comprises a medication.

3. Balloon catheter according to claim 2, wherein the medication is a cytostatic.

4. Balloon catheter according to claim 1, wherein the groove-shaped depressions (4) are configured to run circumferentially on the outside of the balloon (2).

5. Balloon catheter according to claim 4, wherein the groove-shaped depressions (4) run orthogonal to the longitudinal axis of the balloon (2) on the outside of the balloon (2).

6. Balloon catheter according to claim 1, wherein the groove-shaped depressions (4) run in a helical line, in helix shape, on the outside of the balloon (2), wherein the longitudinal axis of the helix corresponds to the longitudinal axis of the balloon (2).

7. Balloon catheter according to claim 1, wherein the groove-shaped depressions (4) run parallel to the longitudinal axis of the balloon (2) on the outside of the balloon (2).

8. Balloon catheter according to claim 1, wherein the groove-shaped depressions (4) run in sine shape on the outside of the balloon (2).

9. Balloon catheter according to claim 1, wherein the depressions (4) have a depth of 1 to 20 μm, preferably of 3 to 8 μm, and particularly preferably of 4 to 6 μm.

10. Balloon catheter according to claim 1, wherein the distance between the depressions (4) amounts to 200 to 400 μm, particularly approx. 300 μm.
